(19) United States
(12) Patent Application Publication
(10) Pub. No.: US 2025/0152372 A1
(43) Pub. Date: May 15, 2025

(54) CONSTRAINED KNEE JOINT PROSTHESIS SYSTEM AND ASSEMBLY

(71) Applicant: Zimmer, Inc., Warsaw, IN (US)

(72) Inventors: Cora Ruth M. Northrop, Claypool, IN (US); David L Glass, Silver Lake, IN (US); Tim Yoko, Granger, IN (US)

(21) Appl. No.: 18/940,255

(22) Filed: Nov. 7, 2024

Related U.S. Application Data
(60) Provisional application No. 63/597,903, filed on Nov. 10, 2023.

(51) Int. Cl. A61F 2/38; A61F 2/30

(52) U.S. Cl. CPC A61F 2/3859; A61F 2/389; A61F 2002/30624

(57) ABSTRACT

A prosthesis system for a constrained knee optionally including: a tibial bearing component configured to articulate with a femoral component; and a hinge post configured to couple with the femoral component and configured to be at least partially received by a well of the tibial bearing component. The hinge post includes one or more ramps and the tibial bearing component includes a riding feature configured to interact with the one or more ramps to rotate the hinge post during distraction of the hinge post from the tibial bearing component.

### CLAIM OF PRIORITY

This application claims the benefit of U.S. Provisional Patent Application Ser. No. 63/597,903, filed on Nov. 10, 2023, the benefit of priority of which is claimed hereby, and which is incorporated by reference herein in its entirety.

### FIELD

The present subject matter relates to orthopedic knee prostheses and, more particularly, to orthopedic knee prostheses and systems for coupling a femoral limb salvage component to tibial baseplate.

### BACKGROUND

Orthopedic procedures and prostheses are commonly utilized to repair and/or replace damaged bone and tissue in the human body. Generally, the knee is formed by the pair of condyles at the distal portion of the femur, the lower surfaces of which bear upon the correspondingly shaped proximal surface plateau of the tibia. The femur and tibia are connected by means of ligaments such as, the posterior cruciate ligament, the lateral collateral ligament, the medial collateral ligament, and the anterior cruciate ligament. These ligaments provide stability to the knee joint.

Prosthetic knee joints can be considered either constrained or unconstrained. For the purposes of this discussion, constrained prosthetic knee systems include femoral and tibial prostheses, which are mechanically linked or constrained to each other to limit relative movement between the femoral and tibial prostheses. Common mechanisms for such mechanical linkage is by a hinge, band or other linkage structure. An unconstrained prosthetic knee system includes femoral and tibial prostheses which are not mechanically linked. An unconstrained knee utilizes the patient's existing ligaments and other soft tissue to provide joint stability. With this in mind, constrained prosthetic knees have particular applicability to cases in which a patient has experienced bone, ligament loss and/or the existing ligaments do not provide adequate support and stability to the knee.

Knee salvage arthroplasty is used to address situations where bone of the femur must be removed and replaced due to a malignancy or other reason. A knee salvage prosthesis system is a particular type of constrained prosthetic knee system. Designs for knee salvage prosthesis systems are known and are commercially available including the OSS™ Orthopedic Salvage System manufactured and sold by the applicant, Zimmer Biomet of Warsaw, Indiana. Many constrained knee designs including the OSS™ Orthopedic Salvage System and NexGen® Rotating Hinge Knee utilize a hinge post. This hinge post configuration is positioned within a tibial baseplate (with an end protruding therefrom) and is connected to the femoral component. However, the design philosophy, shape and degree of constraint of the hinge post and other system components varies from system to system.

### Overview

This disclosure pertains generally to improved constrained knee prostheses, particularly those utilizing a hinge post that can be used with a femoral component such as but not limited to one designed for limb salvage. The femoral component can be part of an existing knee salvage prosthesis system such as the OSS™ Orthopedic Salvage System. The applicant has recently designed a constrained prosthetic knee system with components such as a baseplate that provide for improved stability among other benefits. Examples of the newly designed constrained prosthetic knee system are disclosed in U.S. Provisional Patent Application Nos. 63/434,563, 63/434,573, 63/434,574, 63/434,580, 63/434,584, 63/434,590, 63/450,878 and 63/450,879, the entire specification of each of which is incorporated herein by reference in its entirety. It is therefore desirable that the femoral component such as one designed for limb salvage can be used with the baseplate and other components of the recently designed system. However, making the femoral component of the knee salvage prosthesis system compatible with the recently designed constrained prosthetic knee system presented challenges that were addressed by inventors in the present application. As an example, the knee salvage prosthesis system can utilize a tibial bearing component that is attached to the hinge post and retained against the femoral component. As the articular surface defined by the tibial bearing component rotates (and distracts) with the femoral component via connection with the hinge post, hyperextension is limited by the articular surface connection ultimately to the femoral component. However, the recently designed constrained prosthetic knee system is not constrained in the same manner as that design utilizes a tibial bearing component that is attached to a tibial baseplate not the femoral component. With the recently designed system, the articular surface rotation of the tibial bearing component is driven by femoral conformity. Hyperextension is limited by a bump stop between the femoral component and a yoke adjacent and contacted by the hinge post.

The present inventors were concerned that the femoral component of the salvage system, if not properly constrained, could spin off the tibial bearing component of the recently designed constrained prosthetic knee system. This was a risk with simply combining the designs of the femoral component with the tibial bearing component of the recent system because the femoral component can have flat condyle sections and would not be tethered to the articular surface of the tibial bearing component. With these differences in mind, the present inventors designed a novel hinge post design and a novel tibial bearing component not previously used by either the knee salvage prosthesis system or the recently designed constrained prosthetic knee system. The inventors created these new components to resist spin out so as to maintain stability of the femoral component with the tibial bearing component. Thus, the novel tibial bearing component can include one or more features such as a well and walls that interact with the novel hinge post design to drive articular surface rotation with increased articular surface conformity to the femoral component (as compared with the tibial bearing component of the recently designed constrained prosthetic knee system). The design of the novel hinge post can additionally include one or more ramps that are configured to interact with one or more features of the novel tibial bearing component to return the hinge post into a desired non-distracted position (such as at least partially received by the well) from a distracted and rotated position.

Additional features and benefits of the various examples provided herein will be discussed and/or will be apparent to one of ordinary skill in the art.

To further illustrate the apparatuses and systems disclosed herein, the following non-limiting examples are provided. Parts or all of these examples can be combined in any manner.

Example 1 is a prosthesis system for a constrained knee optionally comprising: a tibial bearing component configured to articulate with a femoral component; and a hinge post configured to couple with the femoral component and configured to be at least partially received by a well of the tibial bearing component; wherein the hinge post includes, one or more ramps and the tibial bearing component includes a riding feature configured to interact with the one or more ramps to rotate the hinge post during distraction of the hinge post from the tibial bearing component.

In Example 2, the subject matter of Example 1 optionally includes, wherein interaction of the one or more ramps with the riding feature orients the hinge post to be at least partially received by the well of the tibial bearing component when distraction is complete.

In Example 3, the subject matter of Examples 1-2 optionally includes, wherein the riding feature is an edge of a protrusion of the tibial bearing component.

In Example 4, the subject matter of Example 3 optionally includes, wherein the protrusion has a half-dome shape and the edge is an arcuate track.

In Example 5, the subject matter of Examples 1-4 optionally includes, wherein the riding feature is one or more surfaces of a recess of the tibial bearing component.

In Example 6, the subject matter of Examples 1-5 optionally includes, wherein the riding feature is positioned on one of a posterior side or an anterior side of the well of the tibial bearing component.

In Example 7, the subject matter of Examples 1-6 optionally includes, wherein the one or more ramps comprises a first ramp spaced laterally from a second ramp relative to a centerline axis of the hinge post.

In Example 8, the subject matter of Example 7 optionally includes, wherein the first ramp and the second ramp are formed by a distal side of a yoke portion of the hinge post, wherein the first ramp and the second ramp are positioned adjacent a proximal end portion of a post of the hinge post.

In Example 9, the subject matter of Examples 1-8 optionally includes, wherein the one or more ramps are angled at least two of: anterior-posterior, medial-lateral and proximal-distal.

In Example 10, the subject matter of Examples 1-9 optionally includes, a femoral component; a baseplate having a distal surface, a proximal surface opposite the distal surface and facing the tibial bearing component, a periphery extending between the proximal surface and the distal surface, a keel extending distally from the distal surface and a recess, wherein the hinge post is configured to be at least partially received by the well of the tibial bearing component and the recess of the baseplate when in a non-distracted position.

Example 11 is a prosthesis assembly for a constrained knee optionally comprising: a tibial bearing component configured to articulate with a femoral component; and a hinge post configured to couple with the femoral component and at least partially received by a well of the tibial bearing component when the hinge post is in a non-distracted position, wherein the hinge post includes, one or more ramps formed by a distal side of a yoke portion of the hinge post, wherein the one or more ramps is configured to interact with the tibial bearing component to return the hinge post into the well from a distracted and rotated position.

In Example 12, the subject matter of Example 11 optionally includes, wherein the tibial bearing component includes a riding feature that interacts with the one or more ramps to rotate the hinge post to a desired position to be received by the well.

In Example 13, the subject matter of Example 12 optionally includes, wherein the riding feature is an edge of a protrusion of the tibial bearing component.

In Example 14, the subject matter of Example 13 optionally includes, wherein the protrusion has a half-dome shape and the edge is an arcuate track.

In Example 15, the subject matter of Examples 12-14 optionally includes, wherein the riding feature is one or more surfaces of a recess of the tibial bearing component.

In Example 16, the subject matter of Examples 12-15 optionally includes, wherein the riding feature is positioned on one of a posterior side or an anterior side of the well of the tibial bearing component.

In Example 17, the subject matter of Examples 11-16 optionally includes, wherein the one or more ramps comprises a first ramp spaced laterally from a second ramp relative to a centerline axis of the hinge post, and wherein the one or more ramps are angled at least two of: anterior-posterior, medial-lateral and proximal-distal.

Example 18 is a hinge post for a constrained knee optionally comprising: a post; a yoke coupled to the post at a distal side thereof, the yoke having an aperture therethrough for receiving a pin that couples the hinge post to a femoral component; and at least a first ramp and a second ramp positioned at the distal side of the yoke adjacent the post, wherein the first ramp and the second ramp are angulated relative to the post and the aperture.

In Example 19, the subject matter of Example 18 optionally includes, wherein the first ramp and the second ramp are both configured to interact with a tibial bearing component to return the hinge post into a well of the tibial bearing component from a distracted and rotated position.

In Example 20, the subject matter of Examples 18-19 optionally includes, wherein the first ramp and the second ramp are positioned on one of an anterior side or a posterior side of a centerline axis of the post.

Example 21 is a tibial bearing component for a constrained knee optionally comprising: an articular surface; a distal surface opposing the articular surface; a posterior periphery; an anterior periphery opposing the posterior periphery; a well recessed from the articular surface and having an aperture therein, wherein the well is configured to receive at least a portion of a hinge post when the hinge post is not distracted from the tibial bearing component; and a riding feature configured to interact with the hinge post to rotate the hinge post during distraction of the hinge post from the tibial bearing component.

In Example 22, the subject matter of Example 21 optionally includes, wherein the riding feature is an edge of a protrusion of the tibial bearing component.

In Example 23, the subject matter of Example 22 optionally includes, wherein the protrusion has a half-dome shape and the edge is an arcuate track.

In Example 24, the subject matter of Examples 21-23 optionally includes, wherein the riding feature is one or more surfaces of a recess of the tibial bearing component.

In Example 25, the subject matter of Examples 21-24 optionally includes, wherein the riding feature is positioned on one of a posterior side or an anterior side of the well of the tibial bearing component.

Example 26 is at least one machine-readable medium optionally including instructions that, when executed by processing circuitry, cause the processing circuitry to perform operations to implement of any of Examples 1-25.

Example 27 is an apparatus comprising means to implement of any of Examples 1-25.

Example 28 is a system to implement of any of Examples 1-25. Example 29 is a method to implement of any of Examples 1-25.

Example 30, the devices and systems of any one or any combination of Examples 1-29 can optionally be configured such that all elements or options recited are available to use or select from.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various examples discussed in the present document.

FIG. 1 is an exploded view of an example of the recently design constrained knee prosthesis system and an example of the knee salvage prosthesis system.

FIG. 2A is a perspective view of a tibial bearing component according to example of the present application.

FIG. 2B is a top plan view of the tibial bearing component of FIG. 2A.

FIGS. 3A and 3B are perspective views of a hinge post according to examples of the present application.

FIGS. 4A and 4B are perspective views showing a ramp of the hinge post interacting with a riding feature of the tibial bearing component to rotate the hinge post (and femoral component coupled thereto) during distraction of the hinge post from the tibial bearing component according to examples of the present application.

FIG. 5 is a cross-sectional view further illustrating interaction of the ramp of the hinge post with the riding feature of the tibial bearing component according to an example of the present application.

FIG. 6 is a side rear perspective view of the interaction of the ramp of the hinge post with the riding feature of the tibial bearing component according to an example of the present application.

FIG. 7 is a perspective view showing the hinge post coupled to the femoral component and with the hinge post fully seated down into a non-distracted position on the tibial bearing component according to an example of the present application.

FIG. 8A is a perspective view of a tibial bearing component according to another example of the present application.

FIG. 8B is a top plan view of the tibial bearing component of FIG. 8A.

FIGS. 9A and 9B are perspective views of a hinge post according to another example of the present application.

FIG. 10A is a plan view from a posterior position showing the hinge post of FIGS. 9A and 9B distracted from the tibial bearing component of FIGS. 8A and 8B according to an example of the present application.

FIG. 10B is a perspective view from an anterior lateral side showing the arrangement of the hinge post and the tibial bearing component from FIG. 10A.

FIG. 11 is a plan view from a proximal position showing an non-distracted arrangement of hinge post of FIGS. 9A and 9B and the tibial bearing component of FIGS. 8A and 8B with the hinge post at least partially received in a well of the tibial bearing component according to an example of the present application.

### DETAILED DESCRIPTION

The present application relates to constrained tibial prosthesis assemblies and systems including a tibial bearing component and a hinge post among other components. This application focuses making a femoral component that is typically constrained to the tibial bearing component compatible with a system that has a baseplate that is attached to the tibial bearing component. Various features, techniques and interactions including those of the hinge post and the tibial bearing component are discussed herein. The present application recognizes that distraction and rotation of the hinge post from the tibial bearing component can occur but provides features of the hinge post and the tibial bearing component that force the hinge post (and the femoral component coupled thereto) to a desired orientation such that the hinge post can return to a non-distracted position. Such non-distracted position can be achieved when the hinge post is at least partially received in a well of the tibial bearing component. Put another way, the features of the hinge post and the tibial bearing component can act to center or orient the hinge post to a desired position relative to the tibial bearing component so as to allow for return of the hinge post back to a desired position relative to tibial bearing component when the hinge post is not distracted. Additionally, the well can act to couple the hinge post to the tibial bearing component (e.g., can constrain rotation of the hinge post relative to the tibial bearing component) when the hinge post is in the non-distracted position. In this manner, the present hinge post and tibial bearing component can prevent undesired roll off of the femoral component from the tibial bearing component and corresponding pain, discomfort and a possible need for medical intervention.

As used herein, the terms “proximal” and “distal” should be given their generally understood anatomical interpretation. The term “proximal” refers to a direction generally toward the torso of a patient, and “distal” refers to the opposite direction of proximal, i.e., away from the torso of a patient. It should be understood that the use of the terms “proximal” and “distal” should be interpreted as though the patient were standing with the knee joint in extension. The intent is to differentiate the terms “proximal” and “distal” from the terms “anterior” and “posterior”. As used herein, the terms “anterior” and “posterior” should be given their generally understood anatomical interpretation. Thus, “posterior” refers to a rear of the patient, e.g., a back of the knee. Similarly, “anterior” refers to a front of the patient, e.g., a front of the knee. Thus, “posterior” refers to the opposite direction of “anterior”. Similarly, the term “lateral” refers to the opposite direction of “medial”. The term “medial-lateral” means medial to lateral or lateral to medial. The term “proximal-distal” means proximal to distal or distal to proximal. The term “anterior-posterior” means anterior to posterior or posterior to anterior.

As used herein, the “periphery” of a tibial bearing component refers to any periphery as viewed in a top plan view, e.g., in a generally transverse anatomical plane. Alternatively, the periphery of a tibial bearing component may be any periphery as viewed in bottom plan view, e.g., in a generally transverse plane and looking at the distal surface adapted to contact a proximal surface of a tibial baseplate.

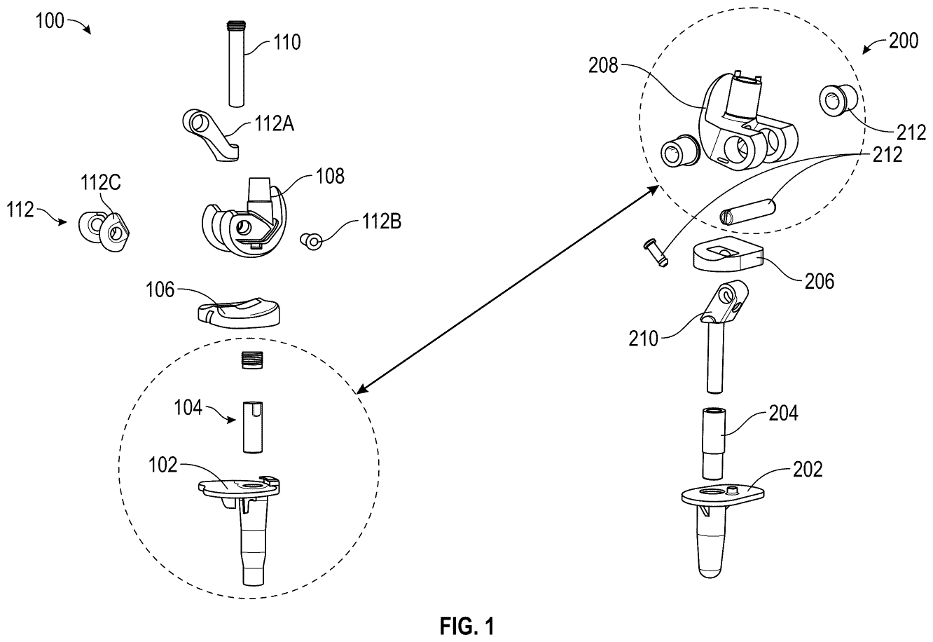

FIG. 1 shows an example prosthesis system 100 for a constrained knee. The prosthesis system 100 can include a tibial baseplate 102, coupling features 104 (e.g., a bushing and/or connection mechanism), a tibial bearing component 106 (sometime called a meniscal component, poly, articular component or bearing), a femoral component 108, a hinge post 110 and connection components 112. FIG. 1 additionally shows a limb salvage system 200. The limb salvage system 200 can include a tibial baseplate 202, coupling features 204 (e.g., a bushing), a tibial bearing component 206, a femoral component 208, a hinge post 210 and connection components 212.

As discussed previously herein, there is a desire to utilize the femoral component 208 of the limb salvage system 200 with the tibial baseplate 102 of the prosthesis system 100. This necessitates design of new components including a new tibial bearing component(s) and new hinge post that can be utilized with the femoral component 208 and the tibial baseplate 102 as previously discussed.

Briefly, the prosthesis system 100 has the femoral component 108 and the tibial baseplate 102 are mechanically linked to one another. This is accomplished by the hinge post 110 and other components illustrated and discussed in briefly herein and in the applicant's prior patent application filings incorporated herein by reference. The hinge post 110 is coupled to the femoral component 108 and is received within a recess of the tibial bearing component 106 and a recess of the tibial baseplate 102. The hinge post 110 is connected to femoral component 108 via the connection components 112 (e.g., a shackle 112A, axle bushing (not shown) and a hinge axle 112B). A distal portion of the shackle 112A can be received in the recess in the tibial bearing component 106 and the distal portion is threaded or otherwise connected to the hinge post 110. The hinge post 110 extends distally through the recess of the tibial bearing component 106 and is received in a recess of the tibial baseplate 102. The recess of the tibial baseplate 102 that receives the hinge post 110 can be at least partially be formed by a keel of the tibial baseplate 102. The hinge post 110 can be moveable (e.g., rotatable and/or capable of distraction) relative to the tibial bearing component 106 and/or the tibial baseplate 102. The hinge post 110 can be rotatably connected to femoral component 108 via the hinge axle 112B. Thus, a longitudinal axis that defines a centerline of the hinge post 110 can define an axis of rotation/articulation for the knee joint as the femoral component 108 and the tibial baseplate 102 are mechanically linked.

When assembled, the shackle 112A can be placed between opposing walls of a poly box 112C. When assembled on the hinge axle 112B, the axle bushing (not shown) additionally resides within an aperture on a proximal portion of the shackle 112A. The shackle 112A and hinge post 110 can be formed from suitable materials such as a titanium alloy, a cobalt-chromium alloy, etc. while the axle bushing and the poly box 112C can be formed from a different materials such as plastic, e.g., UHMWPE. The axle bushing acts as a bearing between the shackle 112A and the hinge axle 112B. The poly box 112C acts as a bearing between the femoral component 108 and the shackle 112A. Prosthesis systems similar to the prosthesis system 100 are described in prior patent filings by the applicant including those incorporated by reference in there entirety above.

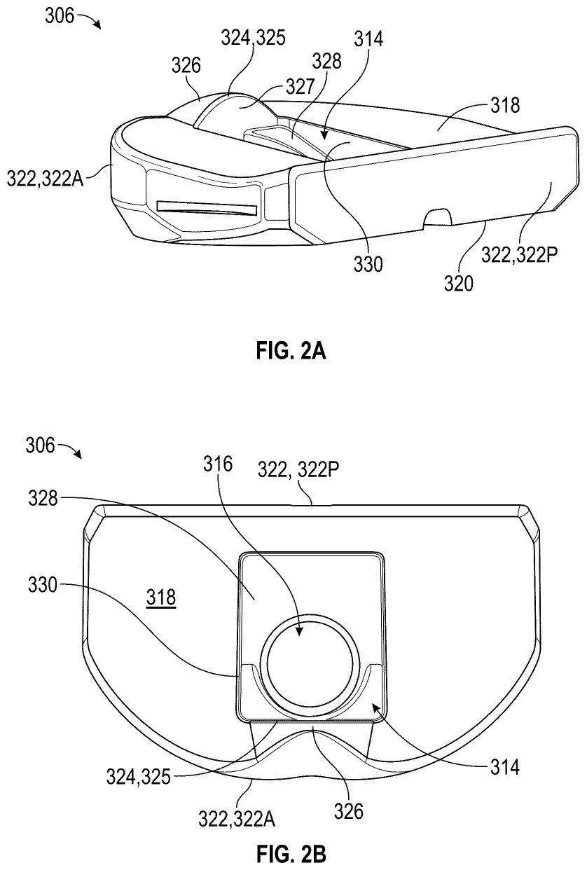

FIGS. 2A and 2B illustrate one example for a tibial bearing component 306 that can be utilized with the portions of the systems 100, 200 described in FIG. 1. The tibial bearing component 306 can include a well 314, an aperture 316 (FIG. 2B), an articular surface 318, a distal surface 320 (FIG. 2A), a periphery 322 including an anterior periphery 322A and a posterior periphery 322P, a riding feature 324 and a protrusion 326. The well 314 can include a distal surface 328 and walls 330 or side surfaces.

The tibial bearing component 306 can be configured to couple with and can be positioned atop a proximal surface of the tibial baseplate 102 (FIG. 1). Such coupling can be via various coupling features known in the art or those previously describe in the applicant's prior filings including those that are incorporated by reference herein. The tibial bearing component 306 can be formed of polymer material such as Ultra-High-Molecular-Weight-Polyethylene (“UHMWPE”), etc. The tibial bearing component 306 can be configured to articulate with the femoral component 208 (FIG. 1) on the articular surface 318 through knee joint flexion and extension as known in the art.

The articular surface 318 can be a proximal surface of the tibial bearing component 306. When assembled, the articular surface 318 can be contacted by condyles of the femoral component 208 (FIG. 1). The distal surface 320 can oppose the articular surface 318. The periphery 322 can extend between the articular surface 318 and the distal surface 320. The anterior periphery 322A can oppose the posterior periphery 322P. The anterior periphery 322A can include relief such as for the patella tendon or other soft tissue. The posterior periphery 322P can be generally flat in extent medial-lateral.

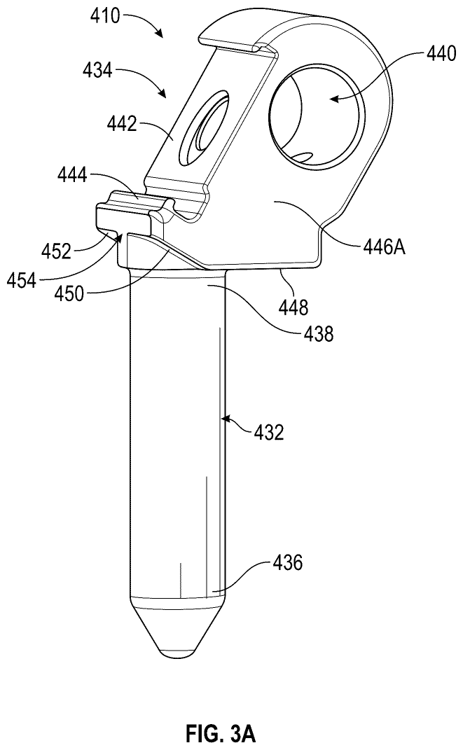

The well 314 can be a box-like square, rectangular or otherwise shaped recess having the distal surface 328 below the articular surface 318. The distal surface 328 of the well 314 can be proximal of the distal surface 320. The distal surface 328 can be generally flat at a posterior portion but can have angled surface(s) so as to receive features of the hinge post at anterior portions thereof. The aperture 316 can extend entirely through the tibial bearing component 306 and can be positioned within the well 314. The walls 330 or side surfaces of the well 314 can extend generally proximal-distal. However, the walls 330 can have some degree of angulation (e.g., additionally extending medial-lateral and/or anterior-posterior) according to some examples. The walls 330 can correspond substantially in shape and size to surfaces of the hinge post (See FIGS. 3A, 3B, etc.) as discussed subsequently. Such design of the well 314, including the walls 330 captures the hinge post to couple the tibial bearing component with the hinge post when the hinge post is in a non-distracted position at least partially received in the well 314. The aperture 316 is configured to receive part of a post of the hinge post when the hinge post is in a non-distracted position at least partially received in the well 314. The aperture 316 can be considered a center of the tibial bearing component 306 as a center of the aperture 316 generally aligns with a centerline axis of a post of the hinge post.

The riding feature 324 can be positioned adjacent the well 314 and can be surface(s), edge(s) or other features of the tibial bearing component 306. In example of FIGS. 2A and 2B, the riding feature 324 can be an edge 325 of the protrusion 326. The protrusion 326 can be in close proximity to the well 314 and can be anterior thereof in the example of FIGS. 2A and 2B. This arrangement positions the riding feature 324 immediately adjacent to and immediately anterior of the well 314. Protrusion 326 can extend proximal of the articular surface 318 such as into a spaced between condyles of the femoral component (not shown) and can be positioned adjacent the anterior periphery 322A. As best shown in FIG. 2A, the protrusion 326 has a half-dome shape with shear or almost vertical posterior surface 327 extending into the well 314. The edge 325 and riding feature 324 is an arcuate track due to the half-dome shape of the protrusion 326.

FIGS. 3A and 3B show a hinge post 410 designed for use with the tibial bearing component 306 of FIGS. 2A and 2B. The hinge post 410 can include a post 432 and a yoke 434. Unlike prior design of the hinge post of the prosthesis system 100 but similar to the limb salvage system 200 of FIG. 1, the hinge post 410 can have the post 432 and yoke 434 integrated as a single component. The post 432 can extend from the yoke 434. The post 432 can have a distal portion 436 and a proximal portion 438. The yoke 434 can have an aperture 440, an anterior side 442, connection features 444, lateral sides 446A (FIG. 3A) and 446B (FIG. 3B), a distal side 448, a first ramp 450 and a second ramp 452.

The yoke 434 can be configured for insertion into a box or other receptacle between the condyles of the femoral component 208 (FIG. 1) and can configured to be at least partially received by the well 314 (FIGS. 2A and 2B) of the tibial bearing component 306. The post 432 can have a shaft like longitudinal extent extending from the yoke 434 and extending between the distal portion 436 and the proximal portion 438. The proximal portion 438 at a proximal end can couple with the yoke 434. Indeed, the post 432 can be formed with the yoke 434 as a single integral component as discussed previously. The proximal portion 438 can be positioned adjacent the first ramp 450 and the second ramp 452 and can be coupled to the distal side 448 of the yoke 434.

The aperture 440 can extend through the yoke 434 from the lateral side 446A to the opposing lateral side 446B. The aperture 440 can be configured to receive a pin or other component of the connection component(s) 212 (FIG. 1). The anterior side 442 can be anterior of the aperture 440 and can extend from the lateral side 446A to the opposing lateral side 446B.

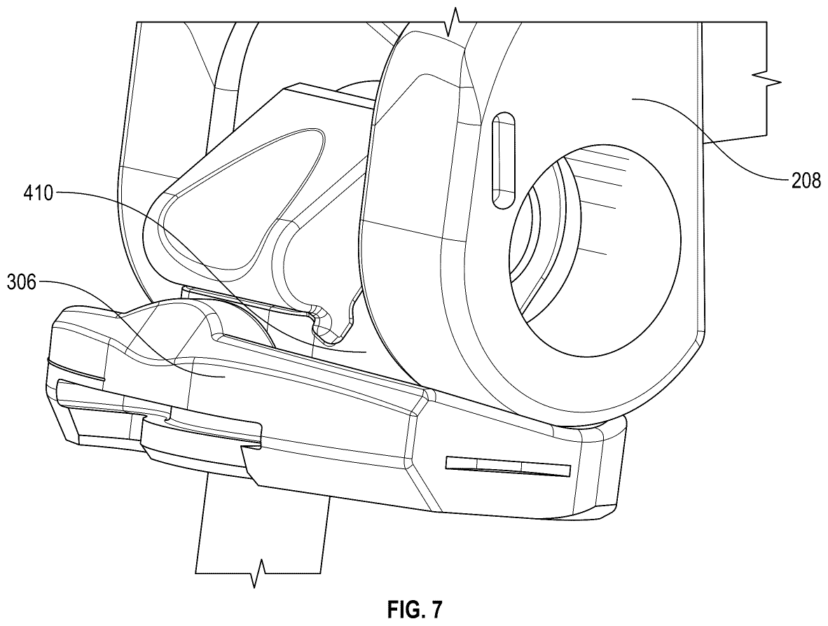

The anterior side 442 can include the connection features 444. The connection features 444 can be configured to receive an extension stop (not shown but shown in FIG. 7) as previously discussed in some of the Provisional patent applications incorporated herein by reference. The extension stop can interact with the femoral component to limit hyperextension of the knee joint as discussed in those prior filings.

The lateral side 446A and the lateral side 446B can be generally flat. However, other examples contemplate the lateral side 446A and the lateral side 446B can be angulated such as medial-lateral. The lateral side 446A and the lateral side 446B extend generally anterior-posterior and proximal-distal when at last partially received in the well 314 (FIGS. 2A and 2B) of the tibial bearing component 306 (FIGS. 2A and 2B). As discussed, the lateral side 446A and the lateral side 446B are sized and shaped to closely correspond to at least some of the walls 330 (FIGS. 2A and 2B) to be spaced therefrom with a small clearance. Interaction of the lateral side 446A and the lateral side 446B with one or more of the walls 330 can limit rotation of the hinge post 410 relative to the tibial bearing component 306 when the hinge post 410 is in the non-distracted position. Thus, the hinge post 410 can be constrained to the tibial bearing component 306.

The distal side 448 can be connected to the lateral side 446A, the lateral side 446B, the anterior side 442 and a posterior side (not shown) of the yoke 434. The distal side 448 can be shaped to correspond with the distal surface 328 (FIG. 2B) of the well 314. Thus, the distal side 448 can include a relatively flat posterior part and the first ramp 450 and the second ramp 452, which are angulated. The first ramp 450 and the second ramp 452 can form an anterior part of the distal side 448. The first ramp 450 can extend from the anterior side 442 to the lateral side 446A. The second ramp 452 can extend from the anterior side 442 to the lateral side 446B. The first ramp 450 and the second ramp 452 can be angled anterior-posterior and proximal-distal. However, other examples contemplate that the first ramp 450 and the second ramp 452 can be additionally angulated medial-lateral. The first ramp 450 can be spaced laterally from the second ramp 452 relative to a centerline axis of the post 432. Thus, the first ramp 450 can terminate prior to reaching the second ramp 452 and can be spaced from the second ramp 452 by a gap 454. The first ramp 450 can have an increasing to decreasing width traveling anterior and laterally. In particular, the width of the first ramp 450 can be largest at a corner between the anterior side 442 and the lateral side 446A but can decrease traveling from this corner more toward the anterior side 442. Similarly, the width can decrease traveling from the corner more toward the lateral side 446A. The second ramp 452 can have a same shape as the first ramp 450 but can be inverted relative to a centerline axis of the post 432.

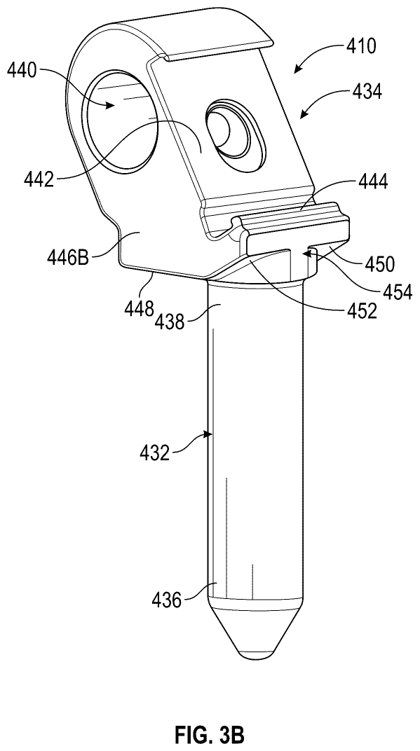

FIGS. 4A and 4B illustrate repositioning, in particular, rotating of the hinge post 410 relative to the tibial bearing component 306 via interaction of the second ramp 452 with part of the riding feature 324 (e.g., the edge 325) of the protrusion 326. As shown in FIG. 4A, the hinge post 410 is distracted from the tibial bearing component 306 and rotated relative thereto. In the rotated position of FIG. 4A, the second ramp 452 contacts the riding feature 324, this contact facilitates rotation (indicated with arrow R1) of the tibial bearing component 306 back toward a centered or desired orientation where the hinge post 410 can be at least partially received by the well 314 so as to return to a non-distracted position. FIG. 4B shows continued rotation (indicated with arrow R1) of the tibial bearing component 306 from the position of FIG. 4A back toward the centered or desired orientation. Put another way, the configuration of the hinge post 410 (using one or both ramps 450, 452) and the configuration of the tibial bearing component 306 (with the riding feature 324) is designed to reverse a clocking of the hinge post 410 relative to the tibial bearing component 306 that can occur during distraction such that the hinge post 410 can return to a non-clocked orientation (so as to be seated down and at least partially within the well 314 once distraction is complete).

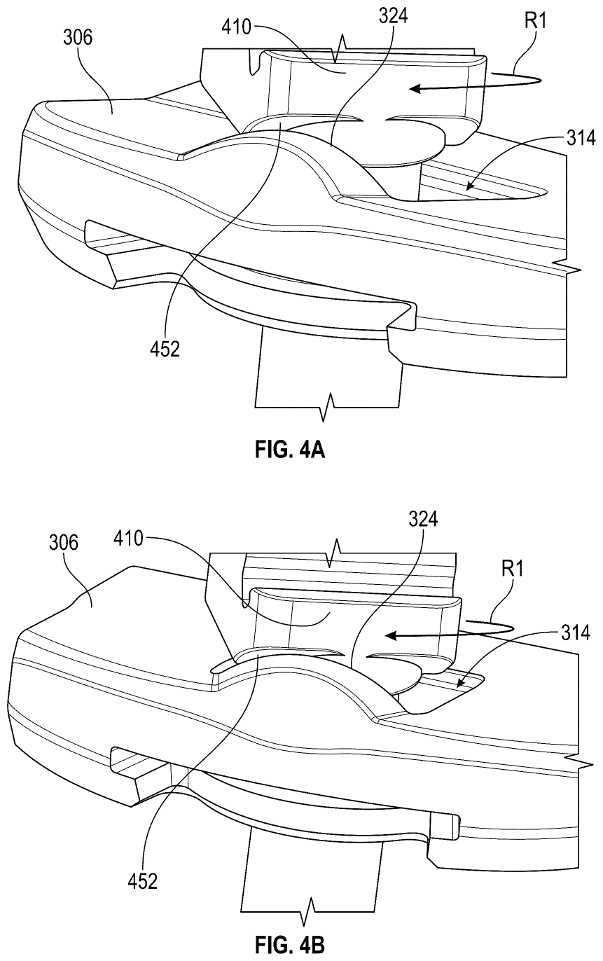

FIG. 5 shows the second ramp 452 interacting (contacting) the riding feature 324 (e.g., the edge 325) of the protrusion 326. As shown in FIG. 5, the edge 325 can be tapered, rounded or otherwise shaped to avoid stress concentration and facilitate sliding movement of the second ramp 452 along the riding feature 324 back toward a position where at least a portion of the yoke 434 can be received by the well 314 once distraction of the hinge post 410 ceases. Once the second ramp 452 no longer contacts the riding feature 324, the yoke 434 and remainder of the hinge post 410 is free to fall into the well 314 as facilitated by the corresponding shape of the well 314 and the yoke 434. Such corresponding shape of the yoke 434 can include the gap 454 (FIGS. 3A and 3B) and the lateral sides 446A and 446B (FIGS. 3A and 3B). The gap 454 can have a small clearance with the posterior surface 327 of the protrusion 326 and the lateral sides 446A and 446B can have a small clearance with some of the walls 330 (FIGS. 2A and 2B).

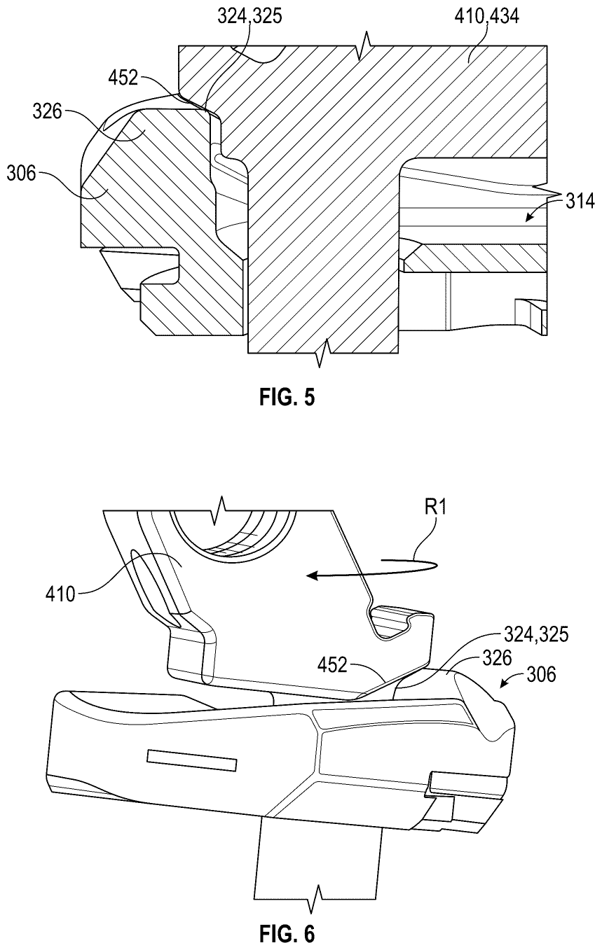

FIG. 6 shows the second ramp 452 interacting (contacting) the riding feature 324 (e.g., the edge 325) of the protrusion 326 from a different perspective than the FIGS. 4A-5. FIG. 6 shows the rotation (indicated with arrow R1) of the hinge post 410 relative to the tibial bearing component 306 via interaction of the second ramp 452 with part of the riding feature 324 to orient the hinge post 410 as desired for being at least partially received by the well (not shown in FIG. 6) when distraction of the hinge post 410 ceases.

FIG. 7 shows the hinge post 410 coupled to the femoral component 208 and seated down in the well (not shown) of the tibial bearing component 306 in the non-distracted position. As discussed previously, when in this non-distracted position, rotation of the hinge post 410 (and the femoral component 208) is constrained by the well of the tibial bearing component 306.

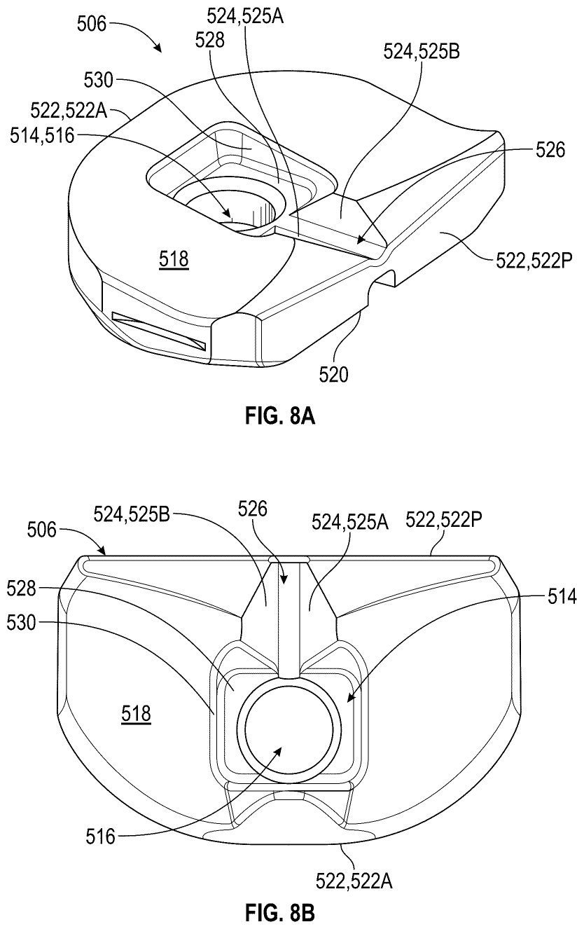

FIGS. 8A and 8B show an alternative example of a tibial bearing component 506 that can be utilized with the portions of the systems 100, 200 described in FIG. 1. The tibial bearing component 506 can include a well 514, an aperture 516, an articular surface 518, a distal surface 520 (FIG. 8A), a periphery 522 including an anterior periphery 522A and a posterior periphery 522P, a riding feature 524 and a recess 526. The well 514 can include a distal surface 528 and walls 530 or side surfaces. The tibial bearing component 506 can be constructed in a manner similar to that of the tibial bearing component 506 previously described in FIGS. 2A and 2B. However, the riding feature 524 and the recess 526 are altered or differ from those previously described.

The riding feature 524 can be formed by the recess 526 and can be positioned posterior of the well 514. The recess 526 can have an inverted groove having a wedge shape and can extend from the well 514 to the posterior periphery 522P, for example. The riding feature 524 can be formed by angulated surfaces 525A and 525B of the recess 526. The surfaces 525A and 525B can be angled medial-lateral and proximal-distal. However, additional examples contemplate the angulated surfaces 525A and 525B can be angled in another manner (e.g., anterior-posterior and proximal-distal).

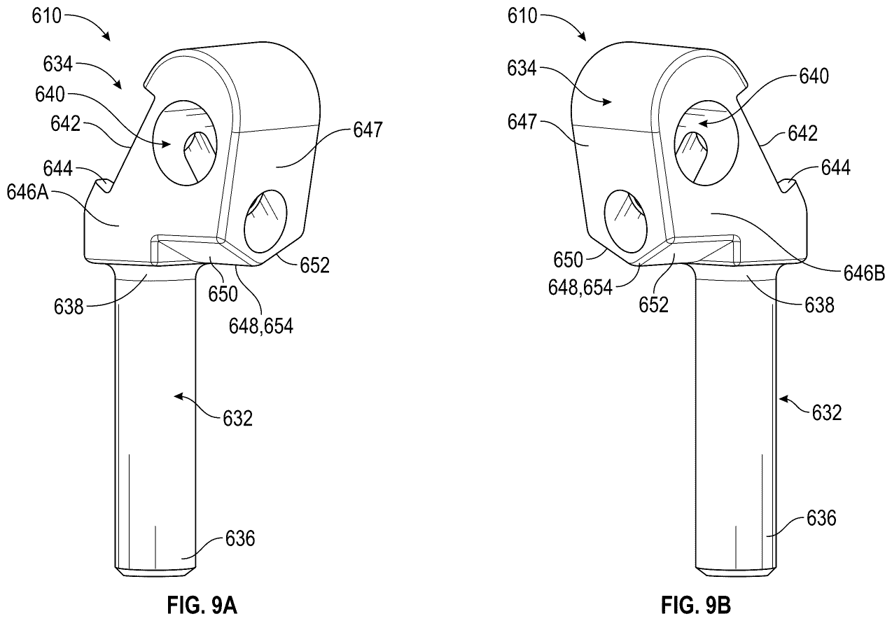

FIGS. 9A and 9B show a hinge post 610 designed for use with the tibial bearing component 506 of FIGS. 8A and 8B. The hinge post 610 can include a post 632 and a yoke 634. The hinge post 610 can have the post 632 and yoke 634 integrated as a single component. The post 632 can have a distal portion 636 and a proximal portion 638. The yoke 634 can have an aperture 640, an anterior portion 642, connection features 644, lateral sides 646A (FIG. 9A) and 646B (FIG. 9B), a posterior side 647, a distal side 648, a first ramp 650 and a second ramp 652. The hinge post 610 can be constructed in a manner similar to that of the hinge post 410 previously described in FIGS. 3A and 3B. However, the distal side 648, the first ramp 650 and the second ramp 652 are altered or differ from those previously described.

The distal side 648 can be connected to the lateral side 646A, the lateral side 646B, the anterior portion 642 and the posterior side 647 of the yoke 634. The distal side 648 can be shaped to correspond with the distal surface 528 (FIG. 8B) of the well 514. Additionally, the distal side 648 can include the first ramp 650 and the second ramp 652 that correspond generally in shape to the recess 526, in particular the surfaces 525A and 525B, respectively (see FIGS. 8A and 8B). The first ramp 650 and the second ramp 652 can form part of a posterior part of the distal side 648. The first ramp 650 can extend from the posterior side 647 to the lateral side 646A. The second ramp 652 can extend from the posterior side 647 to the lateral side 446B. The first ramp 650 and the second ramp 652 can be angled medial-lateral and proximal-distal. However, other examples contemplate that the first ramp 650 and the second ramp 652 can be angulated in another manner such as also anterior-posterior. The first ramp 650 can be spaced laterally from the second ramp 652 relative to a centerline axis of the post 632. Thus, the first ramp 650 can terminate prior to reaching the second ramp 652 and can be spaced from the second ramp 652 by a ridge 654.

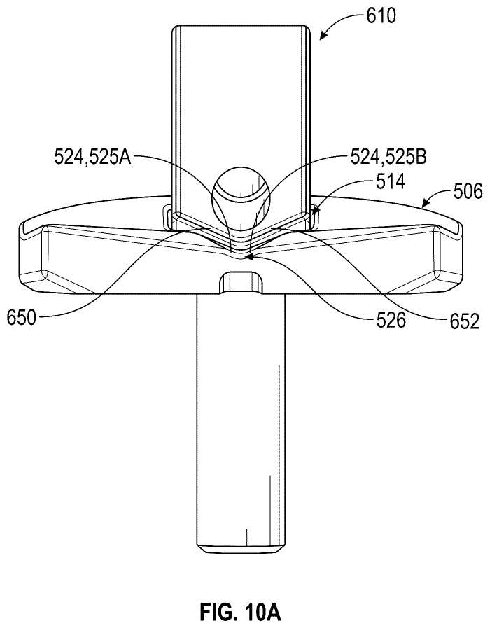

FIGS. 10A and 10B illustrate the hinge post 610 distracted slightly but not rotated relative to the tibial bearing component 506. Should such rotation during distraction occur, interaction of one or both of the first ramp 650 and/or the second ramp 652 (FIG. 10A) with part of the riding feature 524 (e.g., the surface 525A and/or 525B) of the recess 526 (FIG. 10A) act to rotate and reorient the hinge post 610 back toward a centered desired orientation where the hinge post 610 can be at least partially received by the well 514 in the non-distracted position.

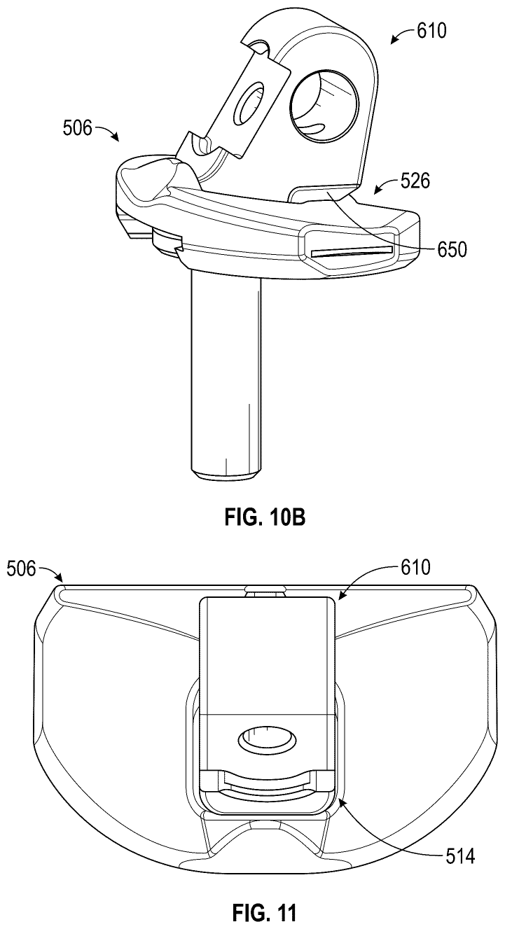

FIG. 11 shows the hinge post 610 partially received by the well 514 of the tibial bearing component 506 in the non-distracted position.

As contemplated therein the ramp(s) and riding feature(s) can be reversed such that the riding feature(s) can be part of the hinge post and the ramp(s) can be part of the tibial bearing component. The terms riding feature(s) and ramp(s) should be interpreted broadly as corresponding features that facilitate a desired movement. As such, the riding feature(s) can be ramp(s) (e.g., be a planar, angled, male feature or female feature) in some examples and the ramp(s) can be a riding feature(s) (e.g., be a planar, angled, female feature or male feature) in some examples. Additionally, such features can be located in different positions from those shown including as parts of the side wall(s) of the well, for example. Additionally, for example, the first ramp 650 and the second ramp 652 could be on an anterior side of the hinge post 610 and the riding feature 524 and the recess 526 could be on the anterior side of the tibial bearing component 506. Alternatively, the first ramp 450 and the second ramp 452 could be on the posterior side of the hinge post 410 and the riding feature 324 and projection 326 could be on the posterior side of the tibial bearing component 306.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “generally” “substantially” “about” mean within 15 percent of the value provided (±). The terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other examples can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above detailed description, various features can be grouped together to streamline the disclosure. This should not be interpreted as intending that an unclaimed disclosed feature is essential to any claim. Rather, inventive subject matter can lie in less than all features of a particular disclosed example. Thus, the following claims are hereby incorporated into the detailed description as examples or embodiments, with each claim standing on its own as a separate example, and it is contemplated that such examples can be combined with each other in various combinations or permutations. The scope of the invention should be determined with reference to the appended claims, along with the full scope of equivalents to which such claims are entitled.

The claimed invention is:

1. A prosthesis system for a constrained knee comprising:
a tibial bearing component configured to articulate with a femoral component; and
a hinge post configured to couple with the femoral component and configured to be at least partially received by a well of the tibial bearing component;
wherein the hinge post includes one or more ramps and the tibial bearing component includes a riding feature configured to interact with the one or more ramps to rotate the hinge post during distraction of the hinge post from the tibial bearing component.

2. The prosthesis system of claim 1, wherein interaction of the one or more ramps with the riding feature orients the hinge post to be at least partially received by the well of the tibial bearing component when distraction is complete.

3. The prosthesis system of claim 2, wherein the riding feature is an edge of a protrusion of the tibial bearing component.

4. The prosthesis system of claim 3, wherein the protrusion has a half-dome shape and the edge is an arcuate track.

5. The prosthesis system of claim 1, wherein the riding feature is one or more surfaces of a recess of the tibial bearing component.

6. The prosthesis system of claim 1, wherein the riding feature is positioned on one of a posterior side or an anterior side of the well of the tibial bearing component.

7. The prosthesis system of claim 1, wherein the one or more ramps comprises a first ramp spaced laterally from a second ramp relative to a centerline axis of the hinge post.

8. The prosthesis system of claim 7, wherein the first ramp and the second ramp are formed by a distal side of a yoke portion of the hinge post, wherein the first ramp and the second ramp are positioned adjacent a proximal end portion of a post of the hinge post.

9. The prosthesis system of claim 1, wherein the one or more ramps are angled at least two of: anterior-posterior, medial-lateral and proximal-distal.

10. The prosthesis system of claim 1, further comprising:
a femoral component;
a baseplate having a distal surface, a proximal surface opposite the distal surface and facing the tibial bearing component, a periphery extending between the proximal surface and the distal surface, a keel extending distally from the distal surface and a recess,
wherein the hinge post is configured to be at least partially received by the well of the tibial bearing component and the recess of the baseplate when in a non-distracted position.

11. A prosthesis assembly for a constrained knee comprising:
a tibial bearing component configured to articulate with a femoral component; and
a hinge post configured to couple with the femoral component and at least partially received by a well of the tibial bearing component when the hinge post is in a non-distracted position, wherein the hinge post includes one or more ramps formed by a distal side of a yoke portion of the hinge post, wherein the one or more ramps is configured to interact with the tibial bearing component to return the hinge post into the well from a distracted and rotated position.

12. The prosthesis assembly of claim 11, wherein the tibial bearing component includes a riding feature that interacts with the one or more ramps to rotate the hinge post to a desired position to be received by the well.

13. The prosthesis assembly of claim 12, wherein the riding feature is an edge of a protrusion of the tibial bearing component.

14. The prosthesis assembly of claim 13, wherein the protrusion has a half-dome shape and the edge is an arcuate track.

15. The prosthesis assembly of claim 12, wherein the riding feature is one or more surfaces of a recess of the tibial bearing component.

16. The prosthesis assembly of claim 12, wherein the riding feature is positioned on one of a posterior side or an anterior side of the well of the tibial bearing component.

17. The prosthesis assembly of claim 11, wherein the one or more ramps comprises a first ramp spaced laterally from a second ramp relative to a centerline axis of the hinge post, and wherein the one or more ramps are angled at least two of: anterior-posterior, medial-lateral and proximal-distal.

18. A hinge post for a constrained knee comprising:
a post;
a yoke coupled to the post at a distal side thereof, the yoke having an aperture therethrough for receiving a pin that couples the hinge post to a femoral component; and
at least a first ramp and a second ramp positioned at the distal side of the yoke adjacent the post, wherein the first ramp and the second ramp are angulated relative to the post and the aperture.

19. The hinge post of claim 18, wherein the first ramp and the second ramp are both configured to interact with a tibial bearing component to return the hinge post into a well of the tibial bearing component from a distracted and rotated position.

20. The hinge post of claim 18, wherein the first ramp and the second ramp are positioned on one of an anterior side or a posterior side of a centerline axis of the post.
